(19) United States
(12) Patent Application Publication
(10) Pub. No.: US 2004/0235183 A1
(43) Pub. Date: Nov. 25, 2004

(54) Assessment of fertility

(76) Inventors: John Coley, Northamptonshire (GB); Fernando Miro, Bedford (GB); Jayne Ellis, Northamptonshire (GB); Paul Henry Charles Mundill, Espoo (FI)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/469,666

(22) PCT Filed: Mar. 4, 2002
(86) PCT No.: PCT/GB02/00923

(51) Int. Cl. G01N 33/48

(52) U.S. Cl. 436/87; 436/65

(57) ABSTRACT

Disclosed is a method of assessing the fertility status of a human female subject, the method comprising the steps of: (a) testing the concentration of FSH in each of a plurality of urine samples obtained from the subject, each sample being obtained on a different day of a first menstrual cycle in the subject; (b) testing the concentration of FSH in each of plurality of urine samples, each sample being obtained on a different day in one or more subsequent menstrual cycles in the subjects; (c) comparing the FSH test results obtained from the subject with a reference value from a control population; and, (d) based at least partly on the comparison, making an assessment of the fertility status of the subject.

### FIELD OF THE INVENTION

This invention relates to a method of assessing the fertility of a human female subject, and to test devices and kits for use in the method, and a monitoring device.

### BACKGROUND OF THE INVENTION

It is well-known that the fertility of women declines from normal levels before the menopause (i.e. cessation of menstruation) is attained. In particular, fertility tends to start to decline markedly beyond the age of about 35, when transition to menopause may commence.

Strictly speaking, fertility is a demographic concept, referring to the percentage of eggs produced by a species or individual which develop into live offspring, whereas fecundity is the term which relates to an individual's capability to conceive (Woods 1989 Oxford Reviews in Reproductive Biology 11, 61-109; Leridon 1977 Levels of natural fertility. In Human Fertility: the basic components. H Leridon ed. Chicago University Press 104-20). However the term fertility has also been widely used with reference to fecundity, and is accordingly so employed in the present specification.

The menopause transition (often referred to as the perimenopause) is considered to extend from the break in cycle regularity through to the post menopause. It is a time of declining fertility, characterised by periods of irregular hormone patterns interspersed with periods of ‘normality’ in which the hormone pattern is indistinguishable with those of a fertile young woman. The latter stages of the perimenopause and the menopause are associated with a high prevalence of hot flushes, a decline in cognitive performance and increased health risks, particularly osteoporosis and cardiovascular disease (CVD). Scientific literature would suggest that it is not possible to identify the menopause at the time it occurs.

The term “ovarian reserve” has been adopted in the art to indicate the remaining fertility timespan for a woman. This is determined largely by the number of structures (follicles) in the ovaries which, when stimulated by pituitary hormones (especially follicle stimulating hormone, abbreviated as FSH) have the capacity to mature and release viable oocytes for possible fertilisation.

At birth the human ovary has between 0.5 and 1.0×10⁶ primordial follicles. No new follicles are formed after birth. However, only a tiny portion (around 400) of these primordial follicles ever mature and ovulate during a woman's reproductive life. The vast majority of follicles undergo atresia (i.e. begin development but never completely mature).

The concentration of FSH at the beginning of the menstrual cycle is believed to be important in ensuring proper maturation of the next dominant follicle. The dominant follicle is selected from the cohort of available follicles by virtue of its higher FSH responsiveness. However this FSH responsiveness will become a problem if the level of FSH increase is too high: a very high concentration of FSH could unbalance the process of proliferation and differentiation of follicular granulosa cells and may result in abnormalities in the process of follicular growth which in turn adversely affect the maturation of the enclosed oocyte, and hence the fertility of the cycle.

Down-regulation of FSH concentration at the beginning of the cycle is achieved through inhibin B, which is produced and secreted by the newly recruited follicles. However, newly recruited follicles are small, and this limits their potential for making inhibin B. In order to overcome the size limitation multiple follicular recruitment is necessary to produce sufficient inhibin B levels, even though at the end, only one follicle will develop to term while the rest degenerate in atresia.

Alternatively stated, FSH is required at the beginning of the menstrual cycle to ‘kick-start’ follicular development, but as soon as this happens the cohort of growing follicles then starts to produce inhibin B, which down-regulates FSH secretion through feedback at the level of the pituitary. This is described in a number of prior art publications e.g.: Illingworth et al, 1991 Journal of Clinical Endocrinology and Metabolism 73:667-73; MacNaughton et al, 1992 Clinical Endocrinology 36:339-45; and Klein et al, 1996 Journal of Clinical Endocrinology and Metabolism 81:2742-5.

However, the number of ovarian follicles in the ovary (‘ovarian reserve’) steadily declines as the woman gets older. This means that the number of recruited follicles in each cycle (i.e., the cohort) cannot be maintained at a sufficiently high number throughout life and in fact, the number of follicles in the cohort decreases in parallel to the decline in ovarian reserve. A reduction in the number of recruited follicles results in an increase in FSH which in turn results in an acceleration in the development of the dominant follicle due to over-stimulation. Menstrual cyclicity is not affected at this stage (apart from a reduction in the length of the follicular phase) and steroid concentrations remain within the normal range and ovulation occurs with regularity. Nevertheless, there is a significant decrease in fertility which is very likely to have its origin in an ineffective maturation of the follicle. As FSH concentration tends to increase during menopausal transition, the process of follicular maturation becomes more and more problematic until eventually even ovulation becomes compromised.

Increasing follicular FSH levels as a consequence of a woman entering the menopause transition is a consequence of decreasing levels of inhibin B and a decline in ovarian reserve, i.e., diminished fertility.

A number of assay methods have been developed in an attempt to measure a woman's ovarian reserve. These include the clomiphene citrate challenge (CCC) test; the GnRH challenge test; and the exogenous FSH test.

In the CCC test, serum FSH on day 2 is measured (day 1 of the cycle being the first day of bleeding), and then 100 mg clomiphene citrate is administered to the subject under investigation on each of days 5-9 (inclusive) of the subject's menstrual cycle. On day 10 the serum FSH level is redetermined. The test result is considered abnormal (i.e. that fertility is impaired) if the FSH level is elevated and/or if the FSH level at day 10 is elevated.

In the GnRH (Gonadotrophin Releasing Hormone) challenge test, subjects are stimulated on day 2 of the cycle with GnRH, following measurement of FSH and estradiol levels. After 24 hours, the estradiol level is re-determined.

In the exogenous FSH test, FSH and estradiol levels are determined before and 24 hours after a single dose of 300 IU of purified FSH administered on day 3 of the menstrual cycle. A level of FSH >10 U/L and an estradiol increase <100 pmolUL are considered abnormal.

One other test routinely used, in predicting the likelihood of a successful outcome following in vitro fertilisation (IVF) treatment, is measurement of serum FSH on a single day. Typically the serum FSH level is measured on day 3 of the cycle. An elevated FSH level (compared to that found in women of the same age with regular cycles) is taken as an indicator of reduced likelihood of success.

All of the above methods suffer from a number of disadvantages. In particular, they are invasive, requiring the taking of blood samples. They are quite expensive to perform, are often analytically unreliable, require the presence of a skilled medical practitioner, and require laboratory analysis to provide the assay result. Accordingly, none of these methods is suitable for use as a routine screening assay.

An improved method of assessing the fertility of a woman, especially of a woman with possibly irregular cycles (frequently of an age of 35 or more) would be a considerable advance, as more women in many countries are delaying starting a family until their mid-thirties or beyond, at which age, fertility is often impaired.

### SUMMARY OF THE INVENTION

In a first aspect the invention provides a method of assessing the fertility status of a human female subject, the method comprising the steps of: (a) testing the concentration of FSH in each of a plurality of urine samples obtained from the subject, each sample being obtained on a different day of a first menstual cycle in the subject; (b) testing the concentration of FSH in each of a plurality of urine samples, each sample being obtained on a different day in one or more subsequent menstrual cycles in the subject; (c) comparing the FSH test results obtained from the subject with a reference value calculated from a control population; and, (d) at least partly based on the comparison making an assessment of the fertility status of the subject.

In some embodiments, the assessment of the fertility status of the subject will be based entirely, or substantially entirely, on the comparison of FSH test results, but in other embodiments additional data (particularly data relating to the urinary concentration of one or more analytes, such as LH) will be employed.

Typically the comparing step (c) will comprise calculating an average urinary FSH concentration from the plurality of tests conducted on the urine samples from the subject.

The reference value with which the subject's urinary FSH concentration is compared may be a value obtained by study of a population of women who are menopausal or, more preferably, compared with a urinary FSH concentration found in women who are of normal fertility and experience regular menstrual cycles.

In particular, the urinary FSH concentration for the subject may be compared to a threshold reference value, an FSH concentration above which is indicative of at least some reduction in fertility (i.e. decreased ovarian reserve) and below which is indicative of normal fertility. The exact value chosen as the threshold reference value will depend on the manner and method used to measure the urinary concentration, for reasons described in greater detail below in the Examples.

The term “assessing the fertility status” as used herein is intended to refer, in particular, to a method of determining whether or not a female subject has a degree of fecundity (i.e. ability to conceive) which is reduced, as a result of the onset of the menopause transition, relative to that of fertile women who have not commenced the menopause transition.

In particular the present inventors have found that, by comparing the average urinary FSH concentration with a particular reference value, they can distinguish between women falling into two groups: a first group consists of women with generally regular cycles and normal fertility; and a second group which consists of all women who have reduced fertility, of varying extent, who have commenced the progression towards menopause. For present purposes, women with normal fertility having regular intercourse are considered to have about an 85% probability of conceiving within 12 months, without medical intervention (see p216, Hatcher et al, “Contraceptive Technology” 17^{th} Revised Edition, Irvington Publishers Inc., N.W., USA), whilst all other women with a lower probability of conceiving are considered to have reduced or diminished fertility. The ability to determine into which of these two groups a particular woman falls will allow the subject to make informed decisions regarding, for example, a) when to start a family, b) which method of contraception is most appropriate, c) lifestyle changes in relation to diet and health, and provide medical practitioners with valuable information in deciding on the most appropriate treatment where couples are experiencing difficulty in conceiving.

Thus, for example, couples experiencing difficulties in conceiving may be reassured if declining ovarian reserve can be excluded as an obstacle to conception.

It is preferred that the urinary concentration of FSH is determined on a plurality of urine samples obtained from the subject during the follicular phase of the menstrual cycle (that is, in the interval spanning from day 1. [i.e. the onset of menses], to the day of ovulation). In particular, it is preferred that the urine samples are taken in the interval spanning days 1-10 of the cycle, more preferably days 1-7, and most preferably days 1-5. Days 1-5 of the cycle may be considered as the “early follicular phase” of the cycle. In a preferred embodiment, the method of the invention comprises determination of a “basal FSH concentration”, being a mean determined from measurements of FSH concentration made on 2 or more (preferably 3 or more, most preferably 4 or more) days in the early follicular phase of the cycle. If desired the basal FSH concentration for a subject may be determined for a single cycle, or (more preferably) may be calculated from data obtained during a plurality of cycles. Preferably sampling is conducted on successive days within each of a plurality of cycles.

Further, the inventors have found that it is preferred to obtain urine samples on at least 3 days per cycle, more preferably samples are obtained on 4 or 5 days per cycle. The inventors have found that more frequent sampling (e.g. six or seven days per cycle), whilst possible and not excluded from the scope of the invention, adds little if anything to the reliability and accuracy of the method of the invention whilst increasing the test burden.

In contrast to, for example, the conventional clomiphene citrate challenge (CCC) test, the method of the present invention requires the measurement of FSH at a plurality of time points in at least two menstrual cycles in a subject, whilst the CCC test is performed within a single menstrual cycle. In addition, it is an essential requirement of the CCC test that an exogenous drug (clomiphene citrate) is administered to the subject, whilst the method of the present invention may be performed entirely without administration of an exogenous substance to the subject.

Accordingly, in a preferred embodiment of the invention, urine samples are obtained for analysis on each of days 1-4 or 1-5 (inclusive) of a plurality of cycles. Preferably the same numerical days of the cycle are chosen for sampling in each of the plurality of cycles.

The inventors have further found that even women with substantially advanced progression towards menopause (and therefore considerably diminished fertility and ovarian reserve) may sporadically experience normal, ovulatory menstrual cycles in which, in theory, conception would be possible. It is therefore an essential feature of the present invention that urinary FSH measurements are made for a plurality of cycles. Preferably FSH measurements are made for samples obtained from at least three cycles. Preferably, but not essentially, the samples are obtained from a plurality of consecutive cycles.

It may be desirable to arrange for the method of the invention to comprise the testing of the concentration of one or more urinary components in addition to FSH, e.g. so as to allow for fluctuations in FSH concentration caused by variations in urine volume. For example, it is known to measure the concentration of creatinine in urine samples as an “internal reference”, since the concentration of urinary creatinine tends to vary mainly with volume of urine produced by the subject.

Alternatively or additionally other urinary components may be tested so as to provide alternative or additional information of use in making the assessment of the fertility status of the subject. In particular, the method of the invention may include testing the urinary concentration of one or more hormones. A preferred example of an additional hormone for urinary concentration testing is luteinising hormone (LH).

In one arrangement, the invention comprises the steps of: (a) testing, separately or in combination, the concentration of both FSH and LH in each of a plurality of urine samples obtained from the subject, each sample being obtained on a different day of a first menstrual cycle in the subject; (b) testing, separately or in combination, the concentration of FSH and LH in each of a plurality of urine samples, each sample being obtained on a different day in one or more subsequent menstrual cycles in the subject; (c) comparing the FSH and LH or combined FSH+LH test results obtained from the subject with reference FSH and LH values, or a reference combined FSH+LH value, as appropriate, calculated from a control population; and (d) based at least partly on the comparison, making an assessment of the fertility status of the subject.

In such an arrangement, the urinary concentration of FSH and LH may be separately determined e.g. using separate assay devices to measure the two analytes, or using a single assay device which is capable of measuring both analytes simultaneously but independently.

However, another embodiment can be envisaged in which a single assay device is used to measure the combined concentration of both FSH and LH (i.e. FSH+LH), (e.g. comprising a reagent which cross-reacts with FSH and LH), to provide a value for the combined concentration of FSH and LH (FSH+LH) without distinguishing the respective contributions to the combined concentration made by FSH and LH.

In an example of such embodiments, the concentration of both FSH and LH, indvidually or in combination, is determined for each of a plurality of urine samples, which may be compared to a threshold reference value, a combined FSH+LH concentration above which is indicative of at least some reduction in fertility (i.e. decreased ovarian reserve) and below which is indicative of normal fertility. As explained elsewhere, the exact value chosen as the threshold reference value will depend on the manner and method used to measure the urinary concentration. In an example described below, the threshold reference value for combined FSH+LH concentration is 9 m IU/ml, and which allows the inventors to distinguish between women falling into a first group with normal fertility, and a second group consisting of all women with reduced fertility as defined above.

Generally, in women with normal fertility, the inventors have found that the combined urinary FSH+LH concentration is less variable than in women with diminished fertility. Further, in women of normal fertility the relative contributions of FSH+LH to the combined urinary FSH+LH concentration are generally rather similar. In contrast, for women of reduced fertility, the contribution of FSH to the combined urinary FSH+LH concentration tends to be greater than that of LH in a statistically significant proportion of the relevant population.

The method of the invention is performed using urine samples from the subject. It is therefore non-stressing, non-invasive and easy to perform. Indeed, in preferred embodiments, the testing may be performed by the subject herself, and therefore does not require a skilled medical practitioner to perform the test.

In principle, any suitable means of determining urinary concentration may be employed in the method of the invention. However, generally preferred are immunological assay techniques, more especially assays of the “immunochromatographic” type, which are well known to those skilled in the art.

A variety of immunoassay techniques are available which enable urine components to be measured. A wide variety of solid phase testing devices such as dipsticks and chromatographic strips have been described in the literature, and can readily be adapted for use in determining urinary FSH. The device should preferably at least be capable of indicating relative levels of FSH in threshold bands. Examples of simple assay technology that can readily be adapted for use in accordance with the method of the invention are described, for example, in EP 0225054, EP 0183442, EP 0186799 and GB 2204398, the disclosures of these specifications being incorporated herein by reference. Disposable assay strips such as those described in GB 2204398 which simply require to be contacted with urine and which provide an assay result in semi-qualitative form, e.g. by means of a series of test zones on the strip which are progressively positive at higher urinary FSH levels, can be used. Multiple strips that respond at different FSH concentrations can be used, rather than a single strip. Preferably, at least one strip corresponds to the threshold FSH reference value. Alternatively, a visually readable quantitative assay can be based on progression of a visible, e.g. coloured, region or “front” over a surface (e.g. radial difflusion), using for example an enzyme-labelled assay.

In a more sophisticated embodiment of the invention, a recording device is provided which incorporates means for reading the result of the urine assay, e.g. by measuring the absorbance by, or fluorescence from, an assay strip. This may enable a more precise numerical indication of FSH concentration to be given, and further enhance the accuracy of the method. Examples of the type of recording device which could be adapted for use in the invention are disclosed in WO 99/51989.

The detailed electronics of a recording device capable of assimilating, remembering and handling analyte concentration data, as well as providing the preferred electronic features of the device discussed herein, and predicting future cycles on the basis of such data, can readily be provided by those skilled in the electronics art once they have been advised of the factors that such a device must take into consideration, and the information that the device must provide for the user. Such detailed electronics do not form part of the invention.

In an embodiment of the invention in which FSH and one or more other urinary components are measured simultaneously, such measurement can if desired be performed using a single testing device, e.g. a device incorporating multiple assay strips, or a single strip capable of independently detecting the level of the different components under test. Alternatively, the FSH and one or more other urinary components can be tested separately, using different testing devices.

The method of the invention may additionally involve a step comprising recording the length of a plurality of cycles in the subject. Conveniently, and desirably, these will be the same cycles as those in which the urine samples are obtained for analysis of FSH. This can readily be accomplished by the subjects under investigation noting the first day of bleeding (i.e. day 1) of each cycle. If desired this information can be provided to a health care professional in electronic form e.g. by storage in the memory of an electronic monitoring device or a PC.

In a second aspect, the invention provides a test kit for use in the method of the first aspect, the kit comprising a plurality of test devices for determining the concentration of FSH in a urine sample from a subject, together with instructions for use in accordance with the method defined above.

Conveniently the test devices will be of the disposable, immunochromatographic type disclosed in the prior art, such as GB 2204398. Conveniently the test devices will be designed and adapted so as to be able to measure the concentration of one or more urinary components (e.g. LH, creatinine) in addition to FSH. Alternatively, the kit may comprise two or more different types of test device: each type of test device being used to test the concentration of a different urinary component.

Desirably the test kit will comprise at least nine test devices, so as to allow a user of the kit to conduct tests on a plurality of samples from a subject over a plurality of cycles (in particular, to allow tests on three samples from each of three cycles). Conveniently the kit will comprise at least 12 test devices, preferably between 15 and 20 test devices.

The test kit may conveniently further comprise a recording means for recording the results of the tests conducted using the test devices. Advantageously the recording means will comprise an electronic memory which, in some embodiments, may be accessed directly or remotely by a clinician or other medically-qualified person to allow for interpretation of the test data. In a preferred embodiment the recording means is incorporated in a monitoring device, as defined below.

The invention further provides, in a third aspect, a monitoring device for use in conjunction with one or more test devices for testing the concentration of FSH in a urine sample from a patient, the monitoring device being suitable for performing the method of the invention. The monitoring device will typically comprise one or more (preferably two or more, more preferably three or more, and most preferably all of the following): receiving means to receive a test device; reading means for reading the results of tests performed using the test devices (which reading means is typically operable when a test device is received in the receiving means); recording means for recording the results of the tests; and processing means to process the results of the tests (e.g. to calculate an average urinary FSH concentration from the test data). The device may further comprise display means to display information obtained from the tests. Conveniently the monitoring device may be supplied as a component of the test kit defined above, but may also be supplied separately. Typically the monitoring device will comprise computer means to interpret the tests results and to conduct processing of the interpreted results. Optionally the monitoring device is designed and adapted so as to interpret and process data from concentration testing of additional urinary components such as LH or creatinine.

A detailed description of suitable test devices, test kits and monitoring devices is provided in WO 99/51989, the content of which is incorporated herein by reference.

The invention will now be described by way of illustrative examples.

### EXAMPLE 1

The inventors gathered data from a confidential study involving a large number of women, aged 30-58, in which daily urine samples were collected over an interval of 6-12 months and stored at 4 to 8° C. (containing sodium azide at 0.1% as preservative) prior to testing.

The samples were analysed to determine the concentration of a number of urinary components, including FSH and LH. Urinary FSH concentration was determined using an immunoassay technique, run on the AutoDELFIA system which is a high throughput automated system designed to operate up to 24 hours a day, with a minimum of operator intervention. Whilst this facilitated handling of the very large number of samples involved, in principle the same basic assay method could be conducted in a non-automated manner.

The particular assay used involved streptavidin-coated plates, a biotin-labelled anti-FSH monoclonal capture antibody (MAb 4882), and a europium (Eu³⁺)-labelled anti-FSH monoclonal (MAb 5948) to generate the assay signal. These antibodies are not essential to performance of the invention: other anti-FSH monoclonals with similar specificities are commercially available, such as FSH-specific clones 6602 and 6601, available from OY Medix Biochemica AB, Finland.

The assay protocol was as follows (Wallac Assay Buffer, Wash Buffer Concentrate and Enhancement Solution are reagents specifically developed for DELFIA assays, and are available from Perkin Elmer Life Sciences [formerly EG & G Wallac] under the respective products codes 1244-111; 1244-114 and 1244-105):

1. Initially, a solution containing biotin-labelled MAb 4882 (at 1/160 dilution) and Eu³⁺ -labelled MAb 5948 (at 1/200 dilution) in Wallac Assay buffer) was prepared and placed in the AutoDELFIA reagent cassette.

2. Streptavidin-coated plates (E.G. & G. Wallac), supplied dry, were loaded into the AutoDELFIA machine and washed with 2×200111 of wash buffer (wash buffer concentrate obtained from E.G. & G. Wallac).

3. Urine samples under test (2511) or standards or controls were dispensed into the wells of the plates.

4. The MAb 4882/MAb 5948 mixture was further diluted 1/100 in assay buffer (from E.G. & G. Wallac) automatically by the AutoDELFIA, giving a final dilution of 1/16,000 for MAb 4882 and 1/20,000 for MAb 5948. 200 μl of the diluted mixture was then added to the wells of the plates.

5. The plate was incubated with shaking for 120 minutes, and then washed with 6×200 μl wash buffer.

6. 2001 μl of enhancement solution (E.G. & G. Wallac) was added to each well, the plate shaken for 5 minutes, and the counts read. The concentration values were calculated from a standard curve using the AutoDELFIA Multicalc programme.

The menstrual cycles of the volunteers in the study were also recorded (e.g. cycle length, etc). Retrospective analysis of the urinary FSH concentration data, and comparison with the menstrual cycles of the volunteers allowed the inventors to separate the women into two distinct groups. A first group with normal fertility—(the “normal fertility” group) had regular cycles of normal duration (defined for the purposes of the study as a mean cycle length of 27.6 days, with a minimum cycle length of 22 days and a maximum cycle length of 35 days) and basal levels of urinary FSH below 5 mIU/ml, as determined by measurements taken on each of days 1 to 5 of their cycles. The second group (the “reduced fertility” group) included all the other women in the study, who had commenced the menopause transition and had progressed to a varying extent towards menopause. At the earliest stage in this progression, the reduced fertility group women had regular cycles but with a slightly shorter follicular phase and a higher basal level of urinary FSH than the normal fertility women.

In particular, statistical analysis enabled the inventors to identify a threshold FSH concentration of 5 mIU/ml. Women whose average urinary FSH concentration during the early follicular phase of their cycle (i.e. days 1-7) was below 5 mIU/ml, according to the AutoDELFIA assay protocol described above, fell into the normal fertility group and had a hormonal output consistent with normal fertility, whereas women whose average urinary FSH concentration was greater than 5 mIU/ml fell into the reduced fertility group and exhibited variability in hormonal output consistent with a level of fertility which was diminished to various degrees.

It will be appreciated by those skilled in the art that using a different assay method, and/or different assay reagents, will produce slightly different values for the absolute FSH concentration. For example, immunoassays do not measure the total amount of FSH present but rather the overall number of epitopes bound by corresponding paratopes in the reaction system. Additionally, the reference preparations against which the assays are calibrated may be different. Thus, for example, the appropriate FSH threshold value when using different assays or reagents might easily be anywhere in the range 3-8 mIU/ml, more especially 4-6 mIU/ml, and the present invention is not restricted to the use of any one particular assay system nor one particular FSH threshold value.

Table 1 below shows a comparison of the ability of urinary FSH determination to distinguish correctly between women with normal fertility and those with reduced fertility of various extent, based on a threshold level of 5 mIU/ml as judged by the AutoDELFIA assay described previously.

The first column indicates the number of cycles over which FSH measurements were made. The second column indicates the number of days per cycle over which measurements were made. The third column indicates the % of women who were correctly assigned as normal fertility group (“NFG”), and the fourth column the % of women correctly assigned as reduced fertility group (“RFG”), for each FSH testing regime. In crude terms, the higher the additive total for % NFG and % RFG in a particular row of the column, the greater the predictive value of the relevant FSH testing regime.

As shown in the bottom three rows of the table, testing urinary FSH on a single day (day 3 of the cycle, taking first day of bleeding as day 1), for either 1, 2 or 3 cycles, gives very poor predictive value and hence little diagnostic information, with many women incorrectly allocated to one of the two groups. The predictive value can be increased marginally by testing FSH on a plurality of days over a single cycle, but the predictive value is still fairly poor.

TABLE 1

| Number of cycles | Number of days | % NFG | % RFG |
|---|---|---|---|
| 3 cycles | 2 | 90.48 | 81.43 |
| (multiple cycles + | | | |
| multiple days) | | | |
| | 3 | 91.27 | 84.69 |
| | 4 | 91.27 | 87.35 |
| | 5 | 88.39 | 89.39 |
| | 6 | 88.10 | 88.98 |
| | 7 | 88.10 | 89.59 |
| 2 cycles | 2 | 90.71 | 76.18 |
| (multiple cycles + | | | |
| multiple days) | | | |
| | 3 | 89.29 | 82.91 |
| | 4 | 88.57 | 86.00 |
| | 5 | 85.00 | 85.64 |
| | 6 | 85.32 | 86.84 |
| | 7 | 85.71 | 87.09 |
| 1 cycle | 2 | 90.26 | 68.83 |
| (single cycle + | | | |
| multiple days) | | | |
| | 3 | 80.52 | 73.86 |
| | 4 | 80.52 | 77.11 |
| | 5 | 81.17 | 78.08 |
| | 6 | 81.17 | 78.25 |
| | 7 | 81.17 | 79.87 |
| 1 cycle | Day 3 | 74.68 | 71.27 |
| 2 cycle | Day 3 | 70.00 | 80.54 |
| (multiple cycles + | | | |
| single day 3) | | | |
| 3 cycle | Day 3 | 69.05 | 84.01 |
| (multiple cycles + | | | |
| single day 3) | | | |

The predictive value of testing on a plurality of days, over 2 cycles is considerably greater and is at a level which provides a useful level of certainty. This is improved still further by testing FSH on a plurality of days over 3 cycles. Maximum predictive value is afforded by testing on 4 or 5 days (during the follicular phase) over 3 cycles. The table also shows that testing for 6 or 7 days over 2 or 3 cycles does not significantly improve the predictive value and would therefore unnecessarily increase the testing burden.

### EXAMPLE 2

Data relating to the urinary concentration of LH were collected as part of the study described above in Example 1. Urinary LH concentrations were determined using the AutoDELFIA system and involved plates coated with anti-LH monoclonal capture antibody (MAb 2119) and use of a europium (Eu³⁺)-labelled anti-LH monoclonal (MAb 2301) to generate the assay signal. These monoclonals are not essential to performance of the invention: other anti-LH monoclonals with similar specificities are commercially available, such as the α-LH specific monoclonal 5501 and the β-LH specific monoclonal 5503, both available from Medix Biochemica Oy, Finland.

The assay protocol was as follows (assay buffer, wash buffer and enhancement solution all as described in Example 1):

LH Assay Method

The assay protocol was as follows (Assay Buffer, Wash Buffer Concentrate and Enhancement Solution are reagents specifically developed for DELFIA assays, and are available from Perkin Life Sciences [formerly EG & C Wallac] under the respective product codes 1244-111; 1244-114 and 1244-105):

1. Initially, a solution containing Eu³⁺-labelled Mab 2301 (at 1/100 dilution) was prepared and placed in the AutoDELFIA reagent cassette.

2. Mab 2119 coated plates (supplied dry), were loaded into the AutoDELFIA.

3. Urine samples under test (25 μl) or standards or controls were dispensed into the wells of the plates.

4. The Eu³⁺-labelled Mab 2301 conjugate was further diluted 1/100 in assay buffer (from E.G. & G. Wallac) automatically by the AutoDELFIA, giving a final dilution of 1/10,000. 200 μl of the diluted conjugate was then added to the wells of the plate.

5. The plate was incubated with shaking for 120 minutes, and then washed with 6×200 μl wash buffer.

6. 200 μl of enhancement solution (E.G. & G. Wallac) was added to each well, the plate shaken for 5 minutes, and the counts read. The concentration values were calculated from a standard curve using the AuioDELFIA Multicalc programme.

As in Example 1, retrospective analysis of the FSH concentration data (from Example 1) in combination with the LH concentration data, enabled the inventors to separate the women into normal fertility and reduced fertility groups. In particular, statistical analysis allowed the inventors to identify a threshold combined total FSH+LH concentration of 9 mIU/ml. Women whose combined average urinary concentration of FSH+LH during the early follicular phase of their cycle was below 9 mIU/ml according to the AutoDELFIA assay protocols detailed above, fell into the normal fertility group and had a hormonal output consistent with normal fertility, whereas women whose combined average urinary concentration of FSH+LH during the early follicular phase exceeded the 9 mIU/ml threshold level fell into the reduced fertility group and exhibited variability in hormonal output consistent with a level of fertility which was diminished to various degrees.

Table 2 below shows the ability of a determination of combined FSH+LH concentration to distinguish between the normal and reduced fertility groups, using a threshold level of 9 mIU/ml. The format of Table 2 follows that for Table 1 above. As is evident from the Table, testing on a number of days during a single cycle gives relatively poor predictive capability, whereas testing on a plurality of days over two or more cycles gives a useful predictive capability.

TABLE 2
MULTIPLE CYCLES + MULTIPLE DAYS
% sequences correctly classified by FSH + LH at
a cut-off of 9 mIU/ml

| Number of cycles | Number of days | % NFG | % RFG |
|---|---|---|---|
| 3 cycles | 2 | 86.72 | 74.09 |
| | 3 | 83.59 | 78.74 |
| | 4 | 85.16 | 79.96 |
| | 5 | 87.50 | 81.58 |
| | 6 | 82.80 | 85.94 |
| | 7 | 81.67 | 85.94 |
| 2 cycles | 2 | 85.92 | 71.35 |
| | 3 | 83.80 | 75.50 |
| | 4 | 84.51 | 77.12 |
| | 5 | 85.21 | 78.20 |
| | 6 | 82.39 | 78.20 |
| | 7 | 80.99 | 79.46 |
| 1 cycle | 2 | 83.33 | 65.42 |
| | 3 | 78.21 | 68.99 |
| | 4 | 80.13 | 71.10 |
| | 5 | 81.41 | 71.27 |
| | 6 | 81.41 | 71.92 |
| | 7 | 81.41 | 73.38 |

Table 3 below compares the predictive capability of FSH concentration determination alone (using a threshold value of 5 mIU/ml) with that of combined FSH+LH concentration determination (using a threshold value of 9 mIU/ml). It is apparent that FSH determination alone is preferred using the particular reagents in question, but it should be borne in mind that, with different reagents, the performance of combined FSH+LH concentration determination might be significantly improved and possibly even surpass that of FSH determination.

TABLE 3
Comparison of FSH versus FSH + LH
for sequences of 3 cycles

| | | % NFG | | % RFG | |
|---|---|---|---|---|---|
| | | FSH | FSH + LH | FSH | FSH + LH |
| Number of cycles | Number of days | 5 mIU/ml | 9 mIU/ml | 5 mIU/ml | 9 mIU/ml |
| 3 cycles | 2 | 90.48 | 86.72 | 81.43 | 74.09 |
| | 3 | 91.27 | 83.59 | 84.69 | 78.74 |
| | 4 | 91.27 | 85.16 | 87.35 | 79.96 |
| | 5 | 88.39 | 87.50 | 89.39 | 81.58 |
| | 6 | 88.10 | 82.80 | 88.98 | 85.94 |
| | 7 | 88.10 | 81.67 | 89.59 | 85.94 |

It will be apparent that, in the Example described above, the FSH and LH concentrations of the urine samples were determined in separate assays, and these results added to give a combined FSH+LH urinary concentration. However, it is also possible to perform a single assay which will simultaneously give a combined FSH/LH concentration valve.

The four hormones FSH, LH, hCG and TSH share certain structural features: each comprises a common α-subunit, whilst a β-subunit is specific to a particular hormone. Accordingly antibodies to the α-subunit tend to cross-react with all four hormones. A pair of anti α-subunit antibodies, directed to different epitopes on the α-subunit, could therefore be used in a sandwich ELISA to detect all four hormones. However, no hCG would be expected to present in a urine sample unless the subject was pregnant. Further, in perimenopausal women the urinary concentration of TSH would normally be so low as to exert a negligible influence on the assay result. Therefore, in practice, such an assay would actually provide a reasonably accurate determination of combined FSH+LH urinary concentration. EP 0 173 341 provides useful guidance in this regard.

A method of identifying antibodies suitable for use in an assay of this type might be as follows:

four different solid phases are prepared by coating the FSH, LH, HCG and TSH onto the polystyrene wells of a microtitre plate at approximately equivalent concentrations. The wells are allowed to come into contact with the different antibodies for a fixed period of time, after which time the wells are washed with buffer. The amount of antibody captured by the various solid phases is measured by addition of an antimouse-alkaline phosphatase enzyme conjugate to the assay wells. After a suitable incubation the wells are washed and enzyme substrate solution added, generating colour in the wells. The optical density (OD) of the coloured solution is measured at 405 nm and is proportional to the amount of antibody captured by the solid phase. Pairs of potentially suitably cross-reacting antibodies can be assayed in competition, to determine whether they bind to different epitopes on the α-subunit.

1. A method of assessing the fertility status of a human female subject, the method comprising the steps of: (a) testing the concentration of FSH in each of a plurality of urine samples obtained from the subject, each sample being obtained on a different day of a first menstrual cycle in the subject; (b) testing the concentration of FSH in each of a plurality of urine samples, each sample being obtained on a different day in one or more subsequent menstrual cycles in the subject; (c) comparing the FSH test results obtained from the subject with a reference value calculated from a control population; and, (d) based at least partly on the comparison, making an assessment of the fertility status of the subject.

2. A method according to claim 1, wherein step (c) comprises deriving an average urinary FSH concentration.

3. A method according to claim 1 or 2, further comprising determining the concentration of LH in each of a plurality of urine samples from the subject.

4. A method of assessing the fertility status of a human female subject, the method comprising the steps of: (a) testing, separately or in combination, the concentration of both FSH and LH in each of a plurality of urine samples obtained from the subject, each sample being obtained on a different day of a first menstrual cycle in the subject; (b) testing, separately or in combination, the concentration of both FSH and LH in each of a plurality of urine samples, each sample being obtained on a different day in one or more subsequent menstrual cycles in the subject; (c) comparing the FSH and LH or FSH+LH test results obtained from the subject with reference FSH and LH values, or a reference combined FSH+LH value, as appropriate, calculated from a control population; and, (d) based at least partly on the comparison, making an assessment of the fertility status of the subject.

5. A method according to any one of the preceding claims, wherein samples in step (a) and/or (b) are obtained in the interval spanning days 1-10 of the menstrual cycle(s).

6. A method according to any one of the preceding claims, wherein samples in step (a) and/or (b) are obtained in the interval spanning days 1-7 of the menstrual cycle(s).

7. A method according to any one of the preceding claims, wherein samples in step (a) and/or (b) are obtained in the interval spanning days 1-5 of the menstrual cycle(s).

8. A method according to any one of the preceding claims, wherein at least two samples in step (a) and/or (b) are obtained on successive days in the cycle(s).

9. A method according to any one of the preceding claims, wherein samples in step (a) and/or (b) are obtained on at least 3 days per cycle.

10. A method according to any one of the preceding claims, wherein samples in step (a) and/or (b) are obtained on 4 or 5 days per cycle.

11. A method according to any one of the preceding claims, wherein step (b) comprises obtaining urine samples on a plurality of days in at least two cycles.

12. A method according to any one of the preceding claims, additionally comprising the step of recording the cycle length of a plurality of cycles in the subject.

13. A method according to any one of the preceding claims, additionally comprising determining the concentration of one or more further urinary components.

14. A method according to claim 13, wherein the concentration of said one or more further urinary components is determined for samples taken on the same days as those samples used for FSH concentration testing.

15. A method according to any one of the preceding claims, wherein the combined concentration of FSH and LH (FSH+LH) is determined.

16. A test kit for use in performing a method according to any one of claims 1-15, the kit comprising a plurality of test devices for determining the concentration of FSH in a urine sample from a subject, and instructions for use of the kit.

17. A kit according to claim 16, additionally comprising a recording means for recording the results of tests conducted using the test devices.

18. A kit according to claim 16 or 17, comprising a plurality of test devices for determining the concentration of LH in a urine sample from a subject.

19. A kit according to any one of claims 16, 17 or 18, wherein the test devices are suitable for determining the concentration of FSH and LH separately or in combination.

20. A monitoring device for assessing the fertility status of a human female subject, the device being adapted for use with a test device for testing the concentration of FSH in a urine sample from the subject, the monitoring device comprising one or more of the following: receiving means to receive a test device; reading means for reading the results of tests performed using the test devices; recording means for recording the results of the tests; processing means to process the results of the tests; and display means to display information obtained from the tests.

21. A monitoring device adapted for use with a kit according to any one of claims 16-19.

22 A monitoring device according to claim 20 or 21, for use in a method in accordance with any one of claims 1-15.
